SEQUENCE LISTING (16 sequences)

SEQ ID NO: 1 - DNA, 12 bp, Artificial Sequence
  A synthetic polynucleotide
aggtcgccgccc

SEQ ID NO: 2 - DNA, 20 bp, Artificial Sequence
  A synthetic polynucleotide
aattccccccccccccaatt

SEQ ID NO: 3 - DNA, 28 bp, Artificial Sequence
  A synthetic polynucleotide
taacccctaacccctaacccctaacccc

SEQ ID NO: 4 - DNA, 22 bp, Artificial Sequence
  A synthetic polynucleotide
ccccaaccccaaccccaacccc

SEQ ID NO: 5 - DNA, 12 bp, Artificial Sequence
  A synthetic polynucleotide
ccctttaacccc

SEQ ID NO: 6 - DNA, 12 bp, Artificial Sequence
  A synthetic polynucleotide
ccctcttaaccc

SEQ ID NO: 7 - DNA, 12 bp, Artificial Sequence
  A synthetic polynucleotide
cccttaatcccc

SEQ ID NO: 8 - DNA, 12 bp, Artificial Sequence
  A synthetic polynucleotide
cctccttcctcc

SEQ ID NO: 9 - DNA, 12 bp, Artificial Sequence
  A synthetic polynucleotide
ccctaactcccc

SEQ ID NO: 10 - DNA, 16 bp, Artificial Sequence
  A synthetic polynucleotide
cccacccaccctccca

SEQ ID NO: 11 - DNA, 12 bp, Artificial Sequence
  A synthetic polynucleotide
ttttcccctttt

SEQ ID NO: 12 - DNA, 10 bp, Artificial Sequence
  A synthetic polynucleotide
cccccccccc

SEQ ID NO: 13 - DNA, 12 bp, Artificial Sequence
  A synthetic polynucleotide
cccccccccccc

SEQ ID NO: 14 - DNA, 20 bp, Artificial Sequence
  A synthetic polynucleotide
cccccccccccccccccccc

SEQ ID NO: 15 - DNA, 12 bp, Artificial Sequence
  A synthetic polynucleotide
tttttttttttt

SEQ ID NO: 16 - DNA, 20 bp, Artificial Sequence
  A synthetic polynucleotide
cccccccccctttttttttt

(19) United States
(12) Patent Application Publication
(10) Pub. No.: US 2015/0110845 A1
(43) Pub. Date: Apr. 23, 2015

(54) ANTIMICROBIAL POLYNUCLEOTIDE-TEMPLATED METAL NANOCLUSTER

(71) Applicant: Agency for Science, Technology and Research, Singapore (SG)

(72) Inventors: Yen Nee Tan, Singapore (SG); Yuan Chong Jason Lim, Singapore (SG); Chao Wang, Singapore (SG); Lian Hui Zhang, Singapore (SG)

(21) Appl. No.: 14/511,240

(22) Filed: Oct. 10, 2014

(30) Foreign Application Priority Data
Oct. 18, 2013 (SG) 201307789-6

(51) Int. Cl. A61K 33/38; A01N 59/16; A61K 9/00; A61K 31/7088; A61K 31/713; A61K 9/14; A01N 43/90; A01N 25/26

(52) U.S. Cl. CPC A61K 33/38; A01N 43/90; A01N 59/16; A01N 25/26; A61K 31/7088; A61K 31/713; A61K 9/14; A61K 9/0014
USPC 424/418; 514/44 R

(57) ABSTRACT

There is provided a polynucleotide-templated metal nanocluster for use in therapy. There is also provided use of a polynucleotide-templated metal nanocluster in the manufacture of a medicament for prophylactically or therapeutically treating a microbial infection as well as a method of prophylactically or therapeutically treating a microbial infection comprising administering to a subject a polynucleotide-templated metal nanocluster. There is also provided an antimicrobial agent comprising a polynucleotide-templated metal nanocluster.

### CROSS-REFERENCE TO RELATED APPLICATION

This application claims the benefit of priority of Singapore patent application No. 201307789-6, filed 18 Oct. 2013, the contents of it being hereby incorporated by reference in its entirety for all purposes.

### TECHNICAL FIELD

The present invention generally relates to a polynucleotide-templated metal nanocluster for use in therapy. The present invention also relates to a method of treating a disease such as a microbial infection using a polynucleotide-templated metal nanocluster.

### BACKGROUND

Antimicrobial agents such as silver have been investigated to determine their therapeutic efficacies, cytotoxicity towards not only a patient, but also towards a disease causative agent as well as the releasability of the antimicrobial agent to a targeted site.

Silver is a therapeutic agent that has been extensively studied. Silver is known to be both an antimicrobial agent and a toxin to humans. These properties arise from the ability of the silver ion to disrupt essential biochemical pathways in both bacteria and human cells. Silver is used in the form of Ag⁺-containing compounds (such as AgNO₃) or silver nanoparticles for antibacterial applications. Although Ag⁺ is extremely potent, they can pose significant problems to human health and well-being when used excessively. For instance, excessive Ag⁺ usage can cause the cosmetically-unappealing condition called Argyria, where the skin turns bluish-grey irreversibly. In addition, excess Ag⁺ can cause cellular oxidative stress and increases one's susceptibility to cancer. Hence, uncontrollable amount of free Ag⁺ in ionic formulations cannot be used as an appropriate antibacterial agent. As an alternative, controlled-release of Ag⁺ in the form of silver nanoparticles has been introduced to address the problem of excessive Ag⁺ cytotoxicity. Typically, silver nanoparticles having dimensions between 10 and 100 nm are used for antimicrobial applications. These silver nanoparticles act as concentrated reserves of Ag⁺, which are transported to biologically-relevant sites and released at a slow rate. However, this slow release of Ag⁺, albeit with low cytotoxicity, renders silver nanoparticles less effective than compounds containing silver ions such as AgNO₃ in antibacterial bioactivity (which in turns suffer from high cytotoxicity).

Recent studies have revealed that most of the silver ions come from oxidation of the zerovalent silver via proton-mediated reactions with dissolved oxygen in the surrounding solution. In order to control the rate of oxidative Ag⁺ controlled-release, the size, morphology and surface chemistry of silver nanoparticles are normally targeted for modification. Changing the particle size and/or morphology of the nanoparticles vary the total exposed surface area for oxidation by oxygen. Surface chemistry variation, which modifies the silver nanoparticle formation with the use of different stabiliser molecules, control the access of oxygen to the surface of the silver nanoparticle. Some of these stabilisers, such as the commonly-used thiols, can bind very strongly to the surface of the silver nanoparticles, preventing Ag⁺ release through surface passivation and thus negating their antibacterial effects. Other than their difficulty of handling (thiols are very foul-smelling), modifying the chemical structure of these stabilisers will not result in a significant change in Ag⁺ release rate due to the intrinsically-strong Ag-stabiliser binding. These means of control are haphazard, unsystematic, and can be tedious to achieve, often leading to polydispersity in term of nanoparticle size and morphology during the synthesis.

Alternatively, reactive oxygen-containing species responsible for oxidation of silver nanoparticles can be removed using chemicals such as antioxidant enzymes. However, such formulations are cumbersome, difficult to fine-tune and control, and may also pose cytotoxicity issues due to the toxic nature of these chemicals.

Furthermore, despite the above attempts to control the Ag⁺ release rate, no present means exist to allow easy monitoring of the process without requiring complex and specialised techniques such as the graphite-furnace atomic absorption spectroscopy. This can lead to a dangerous false sense of security as the end-user cannot easily assess the actual potency of the silver nanoparticles against bacteria.

Although silver nanoparticles have been used extensively as antibacterial agents, while they are able to inhibit the growth of bacteria, they are unable to prevent the production of harmful toxins from the bacteria. Unless the inhibition of toxin production is achieved, the mere inhibition of bacterial growth will not negate the inherent danger of toxins as toxins can still be produced by any bacteria still present. There are currently no silver-based compounds, including Ag⁺ and silver nanoparticles, which have been reported to inhibit toxin production. Therefore, although these silver-based agents can inhibit bacterial growth, they do not yet offer complete protection against the dangers posed by multidrug-resistant bacteria.

While drugs that are capable of inhibiting toxin production exist, they are organic molecule-based antibiotics which pose formidable synthetic challenges and therefore cannot be implemented on large scales at low cost.

There is a need to provide a metal-based therapeutic agent that overcomes, or at least ameliorates, one or more of the disadvantages described above.

There is a need to provide a metal-based therapeutic agent with a controlled and desired release rate which can be conveniently and unambiguously assessed.

There is a need to provide a metal-based therapeutic agent which not only inhibits bacterial growth, but also inhibits bacterial toxin production.

### SUMMARY

According to one aspect, there is provided a method for inhibiting the growth of or killing microorganism that is present on a surface comprising the step of administering a polynucleotide-templated metal nanocluster to said surface.

Advantageously, the polynucleotide-templated metal nanocluster may release its metal (typically in the form of metal ions) at a rate which can be controlled and which has a therapeutic or antimicrobial effect. The release rate may not excessively increase the amount of metal ions to the extent that they become harmful or toxic to humans and the environment.

Advantageously, the polynucleotide-templated metal nanoclusters, such as DNA-templated silver nanoclusters, exhibit at least one of photoluminescent, antimicrobial and toxin inhibition properties.

Advantageously, the polynucleotide sequences forming the template are obtainable at low cost, are customizable and need not be harvested from any living organism. Hence the polynucleotide-templated metal nanoclusters may be safe and reliable for use across a wide variety of both in-vivo and in-vitro applications.

The DNA-templated silver nanoclusters enjoy a number of advantages as compared to existing thiol-based silver nanoparticles or AgNO₃.

Firstly, the release rate of Ag⁺ from DNA-templated silver nanoclusters may be easily controlled by varying the DNA template sequence in a systematic manner. This can be achieved by simply altering the structure of the DNA surrounding the silver nanoclusters to change the DNA-silver nanocluster surface binding interactions, thereby affecting the Ag⁺ release rate.

Secondly, the DNA template sequences are totally biocompatible, easy to handle and odourless, unlike thiols.

Thirdly, the Ag⁺ release rates can be systematically and easily tuned, unlike the haphazard random chemical modification of thiols for the same purpose.

Fourthly, the DNA-templated silver nanoclusters are responsive to the spontaneous oxidative release of Ag⁺ with intrinsic fluorescent colour changes, which allows an end-user to track the rate and extent of Ag⁺ release easily with readily-available analytical tools and without much technical expertise. In addition, by simply changing the DNA template for AgNC formation, the rate of Ag⁺ release can be easily tuned. This is not possible with any of the existing Ag⁺ controlled-release formulations (silver nanoparticles or AgNO₃) available.

Fifthly, the DNA-templated silver nanoparticles may have stronger antibacterial effects than silver nanoparticles. In addition, the DNA-templated silver nanoparticles are able to inhibit both bacterial growth and bacterial toxin production, as compared to just inhibition of bacterial growth only with the use of silver nanoparticles. This effectively removes the need for expensive additional chemicals to inhibit bacteria toxin production, thereby enabling the DNA-templated silver nanoclusters to function as a single-application antibacterial agent capable of an all-round defence against the dangers posed by bacteria.

Lastly, the DNA-templated silver nanoclusters are not as cytotoxic as AgNO₃. Hence, the DNA-templated silver nanoclusters can be used as a controlled-release therapeutic agent that has both antibacterial performance and biocompatibility.

According to a further aspect, there is provided an antimicrobial agent comprising a polynucleotide-templated metal nanocluster.

### Definitions

The following words and terms used herein shall have the meaning indicated:

The term “metal nanocluster” is to be interpreted broadly to refer to an agglomeration of at least two (zero-valent) metal atoms, metal ions, metal complexes or mixtures thereof (as the case may be), forming a cluster having a dimension of less than 2 nm, less than 1.5 nm, less than 1 nm, less than 0.5 nm or less than 0.1 nm. The dimension of the metal nanocluster may be regarded as the diameter of the metal nanocluster (for a spherical particle) or be regarded as the equivalent spherical diameter of the metal nanocluster (for an irregularly shaped particle). The metal nanocluster may or may not emit a fluorescent signal. The fluorescent signal may be a fluorescent colour that can be detected by a naked eye or with a fluorescent spectrometer of any degree of sensitivity.

Where the metal nanoclusters are supported or encapsulated by a polynucleotide, the resultant particle may be termed as “polynucleotide-templated metal nanoclusters”. The polynucleotide may serve as a scaffold to support and stabilize the metal nanoclusters while minimizing aggregation of the metal nanoclusters with each other. Hence, the polynucleotides aid in maintaining the size of the nanoclusters. By altering the strand length, base sequence or secondary structure of the polynucleotide, different polynucleotide-templated metal nanoclusters can be created with fluorescence emission bands ranging from the near ultraviolet to the visible to the near-infrared range. Where the polynucleotide is a DNA, the polynucleotide-templated metal nanocluster may be termed as a DNA-templated metal nanocluster.

The term “polynucleotide” is to be interpreted broadly to refer to a polymeric form of nucleotides of any length, such as ribonucleotides, deoxyribonucleotides or peptide nucleic acids (PNAs), that comprise purine and pyrimidine bases, or other natural, chemically or biochemically modified, non-natural, or derivatized nucleotide bases. As used herein, the length of the polynucleotide may be at least 2 nucleotides (or 2-nucleobases). The upper limit of the polynucleotide is not specifically restrictive and polynucleotide sequences of any length can be used. In one embodiment, the polynucleotide sequence can range from 2 to 500 nucleotides (or 2-nucleobases to 500-nucleobases). The polynucleotide may be double stranded or single stranded and may contain any supramolecular structures including but not limited to loops and quadruplexes. References to single stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof.

The term “treatment” is to be interpreted broadly to refer to any and all uses which remedy a disease state or symptoms, inhibit or prevent the establishment of disease, or otherwise prevent, hinder, retard, or reverse the progression of disease or other undesirable associated symptoms in any way whatsoever.

The term “therapeutically effective amount” is to be interpreted broadly to include a sufficient but non-toxic amount of a particle or formulation to provide a desired therapeutic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

Exemplary, non-limiting embodiments of a polynucleotide-templated metal nanocluster will now be disclosed.

The polynucleotide-templated metal nanocluster may be used in therapy. The polynucleotide-templated metal nanocluster may be used as a medicament for the prophylactic or therapeutic treatment of a microbial infection. The polynucleotide-templated metal nanocluster may be used in a method of prophylactically or therapeutically treating a microbial infection. The polynucleotide-templated metal nanocluster may be used in a method for inhibiting the growth of or killing microorganism that is present on a surface. The microbial infection may be a bacterial infection, viral infection or fungal infection. Hence, the microorganism may be a bacteria, fungi or virus.

In the polynucleotide-templated metal nanocluster, the polynucleotide may be a DNA, RNA or mixtures thereof. Where the polynucleotide is a DNA, the DNA may be a single-stranded DNA or double-stranded DNA. The DNA may be selected from the group consisting of homocytosine single-stranded DNA sequence (such as C₁₀, C₁₂, or C₂₀), homoadenine single-stranded DNA sequence, homothymine single-stranded DNA sequence (such as T₁₂), homoguanine single-stranded DNA sequence, C₁₀T₁₀, 5′-AGGTCGCCGCCC-3′, 5′-AATTCCCCCCCCCCCCAATT-3′, i-motif (dTA₂C₄)₄, i-motif (dC₄A₂)₃C₄, 5′-CCCTTTAACCCC-3′, 5′-CCCTCTTAACCC-3′, 5′-CCCTTAATCCCC-3′, 5′-CCTCCTTCCTCC-3′, 5′-CCCTAACTCCCC-3′, 5′-CCCACCCACCCTCCCA-3′, dT₄C₄T₄, dsDNA with a C₆ loop and mixtures thereof. It is to be noted that any combination of the nucleotide (A, T, G, C) of any length can be used in the polynucleotide sequence. Hence, each nucleotide may appear singly or consecutively in a polynucleotide sequence or may not appear at all.

The length of the polynucleotide may be at least 2-nucleobases. As mentioned, the length of the polynucleotide may not be specifically restrictive. In one embodiment, the length of the polynucleotide may be in the range of about 2-nucleobases to about 500-nucleobases, about 2-nucleobases to about 400-nucleobases, about 2-nucleobases to about 300-nucleobases, about 2-nucleobases to about 200-nucleobases, about 2-nucleobases to about 100-nucleobases, about 2-nucleobases to about 50-nucleobases, about 2-nucleobases to about 40-nucleobases, about 2-nucleobases to about 30-nucleobases, about 2-nucleobases to about 20-nucleobases, about 2-nucleobases to about 10-nucleobases, about 2-nucleobases to about 5-nucleobases, about 5-nucleobases to about 500-nucleobases, about 10-nucleobases to about 500-nucleobases, about 20-nucleobases to about 500-nucleobases, about 30-nucleobases to about 500-nucleobases, about 40-nucleobases to about 500-nucleobases, about 40-nucleobases to about 500-nucleobases, about 50-nucleobases to about 500-nucleobases, about 100-nucleobases to about 500-nucleobases, about 200-nucleobases to about 500-nucleobases, about 300-nucleobases to about 500-nucleobases or about 400-nucleobases to about 500-nucleobases. The length of the polynucleotide may be about 2-nucleobases, about 10-nucleobases, about 12-nucleobases, or about 20-nucleobases.

The metal of the metal nanocluster may be a noble metal. The metal may be selected from the group consisting of silver, gold, copper, palladium, ruthenium, rhodium, osmium, iridium, platinum, rhenium, mixtures and complexes thereof. The metal of the metal nanocluster may be silver.

The polynucleotide-templated metal nanocluster may comprise more than one type of metal. The polynucleotide-templated metal nanocluster may additionally or alternatively comprise more than one type of polynucleotide. The polynucleotide-templated metal nanocluster may additionally or alternatively comprise the same type of polynucleotide but with varying base sequences and/or lengths.

The metal nanoclusters can be used as mixtures of nanoclusters from different DNA sequence templates (e.g. mixture of C10-silver nanoclusters with C20-silver nanoclusters). The color of the nanocluster mixture can be quantified by an emission color ratio (CR) calculated based on the quotient between the sum of green and blue fluorescence intensities relative to the red fluorescence intensity, or

$$C R value = \frac{\begin{matrix}Green ( {λ}_{nm} = 540 nm ) + \\ Blue ( {λ}_{nm} = 490 nm ) fluorescence intensity\end{matrix}}{Red \{ {λ}_{nm} = 640 nm ) fluorescence intensity} .$$

The nanocluster may have a particle size of about 0.01 nm to about 2 nm, about 0.01 nm to about 1.5 nm, about 0.01 nm to about 1 nm, about 0.01 nm to about 0.5 nm, about 0.01 nm to about 0.1 nm, about 0.1 nm to about 2 nm, about 0.5 nm to about 2 nm, about 1 nm to about 2 nm or about 1.5 nm to about 2 nm. The particle size of the metal nanocluster may be less than about 2 nm.

The metal nanoclusters may be encapsulated by the polynucleotide template. The metal nanoclusters may have tunable photoluminescent, antimicrobial and/or toxin inhibition properties. The polynucleotide-templated metal nanocluster may emit a fluorescence colour that may be dependent on the sequence, structure and length of the polynucleotide template. The fluorescence may be in the near-ultraviolet, visible wavelength or near-infrared spectrum. A color change means a shift in the fluorescence wavelength. For example, due to an oxidation reaction caused by atmospheric oxygen, a color change phenomenon termed as “fluorescence blue-shift” may occur. The opposite effect, termed as “fluorescence red-shift”, may occur due to a reduction reaction.

The ultrasmall metal nanoclusters such as silver nanoclusters that are encapsulated by a polynucleotide template such as a DNA template may be able to release Ag⁺ spontaneously via an oxidation reaction, a process which termed as “spontaneous oxidative release”.

The polynucleotide-templated metal nanoclusters may be formed by chemical reduction of a polynucleotide-complexed metal ion. Where the metal is silver, the polynucleotide-templated silver nanocluster may be formed through chemical reduction by a reducing agent (such as sodium borohydride) in aqueous solution based on the high affinity of Ag⁺ with a nucleotide such as cytosine.

Due to the small size of the metal nanocluster, the total exposed surface area of the metal nanocluster is large enough such that the rate of metal ion controlled-release is greater than that of larger sized metal nanoparticles (where the particle size is more than 10 nm).

The polynucleotide-templated metal nanocluster may be used in therapy. The polynucleotide-templated metal nanocluster may be used in the treatment of a microbial infection. The treatment may be a topical treatment of a microbial infection.

There is also provided the use of a polynucleotide-templated metal nanocluster in the manufacture of a medicament for prophylactically or therapeutically treating a microbial infection. The medicament may be formulated for topical administration.

There may be provided a method of prophylactically or therapeutically treating a microbial infection comprising administering to a subject a polynucleotide-templated metal nanocluster. The administration may be a topical administration.

The polynucleotide-templated metal nanocluster may also be used to inhibit or kill microorganisms that may be present on a surface. The polynucleotide-templated metal nanocluster may be used to disinfect or sterilize a surface in which sterility is essential or desired such as in a laboratory environment (for example, laboratory workbench or laboratory equipment), in a medical environment (for example, exposed surfaces in a surgery room, medical devices used in or on a human or animal body, clinics, hospitals etc), in general disinfection of surfaces that can be exposed to microorganisms (for example, in homes, schools, offices, vehicles, etc) or on commonly used objects such as clothes, accessories or shoes. It is to be noted that such examples are not exhaustive and the polynucleotide-templated metal nanocluster can be used to treat any surface to inhibit bacterial growth or to kill microorganism that may already be present on the surface.

Hence, there is provided a method for inhibiting the growth of or killing microorganism that is present on a surface comprising the step of administering a polynucleotide-templated metal nanocluster to said surface.

The surface may include a skin surface of a human or animal body or may include a surface of an object, such as those mentioned above.

The microbial infection may be a bacterial infection. The bacterial infection may include any infections caused by any bacteria. The bacterial infection may be selected from the group consisting of a Gram positive bacterial infection and a Gram negative bacterial infection.

Exemplary types of bacteria that may cause a bacterial infection or which may be present on a surface may be of a genus including, but not limited to, Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Propionibacterium, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp.

The bacteria that may cause a bacterial infection or which may be present on a surface may be a gram-positive or gram-negative bacteria including, but are not limited to, Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Propionibacterium acnes, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis.

The Gram negative bacteria that may cause a bacterial infection or which may be present on a surface may be Pseudomonas aeruginosa or Burkholdera cenocepacia.

When the bacteria is capable of causing a bacterial skin infection that can be treated topically by the polynucleotide-templated metal nanocluster, the bacterial skin infection may be selected from the group consisting of impetigo (such as nonbullous impetigo), folliculitis, acne, infections of burn injuries and otitis externa. These bacterial skin infections are then caused by Streptococcus pyogenes, Staphylococcus aureus, Pseudomonas aeruginosa, Propionibacterium acnes or Pseudomonas aeruginosa.

The Gram negative bacteria that may cause a bacterial infection may be Pseudomonas aeruginosa or Burkholdera cenocepacia.

When used in therapy or as a medicament, the polynucleotide-templated metal nanocluster may be administered or dosed at a concentration of at least about 150 nM, at least about 180 nM, at least about 200 nM, at least about 250 nM, at least about 300 nM, at least about 350 nM, at least about 400 nM, at least about 450 nM, at least about 500 nM, at least about 550 nM, at least about 600 nM, at least about 650 nM, at least about 700 nM, at least about 750 nM, at least about 800 nM, at least about 850 nM, at least about 900 nM, at least about 950 nM, or at least about 1000 nM.

The polynucleotide-templated metal nanocluster may be able to inhibit bacterial growth and/or inhibit bacterial toxin production. The polynucleotide-templated metal nanocluster may be able to inhibit at least about 20%, at least about 30%, at least about 40%, at least about 50%, at least about 60%, at least about 70%, or at least about 80% of bacterial growth. The polynucleotide-templated metal nanocluster may be able to inhibit at least about 20%, at least about 30%, at least about 40%, at least about 50%, at least about 60%, at least about 70%, at least about 80%, at least about 90%, at least about 95%, at least about 96%, at least about 97%, at least about 98% at least about 99%, or about 100% of bacterial toxin production.

The polynucleotide-templated metal nanocluster may be administered or packaged as a pharmaceutical composition or formulation. The pharmaceutical composition or formulation may be topically administered for local treatment.

Compositions as described herein include, but are not limited to, solutions, emulsions, and liposome-containing formulations. These compositions may be generated from a variety of components that include, but are not limited to, preformed liquids, self-emulsifying solids and self-emulsifying semisolids.

The formulations as described herein may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The pharmaceutical composition may be formulated into any of many possible dosage forms including, but not limited to creams, emulsions, suspensions, ointment or soft gels. The pharmaceutical composition as described herein may also be formulated as suspensions in aqueous, non-aqueous or mixed media. Aqueous suspensions may further contain substances that increase the viscosity of the suspension including, for example, sodium carboxymethyl cellulose, sorbitol and/or dextran. The suspension may also contain stabilizers.

The pharmaceutical composition may be formulated and used as foams. Pharmaceutical foams include formulations such as, but not limited to, emulsions, microemulsions, creams, jellies and liposomes. While basically similar in nature these formulations vary in the components and the consistency of the final product.

The pharmaceutical composition may additionally contain other adjunct components conventionally found in a pharmaceutical composition. Thus, for example, the pharmaceutical composition may contain additional, compatible, pharmaceutically-active materials such as, for example, antipruritics, astringents, local anesthetics or anti-inflammatory agents, or may contain additional materials useful in physically formulating various dosage forms of the pharmaceutical composition, such as dyes, flavoring agents, preservatives, antioxidants, opacifiers, thickening agents and stabilizers. However, such materials, when added, should not unduly interfere with the biological activities of the polynucleotide-templated metal nanocluster of the pharmaceutical composition. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the polynucleotide-templated metal nanocluster of the formulation.

Dosing is dependent on severity and responsiveness of the disease state to be treated, with the course of treatment lasting from several days to several months, or until a cure is effected or a diminution of the disease state is achieved.

The subject may be an animal, mammal, human, including, without limitation, animals classed as bovine, porcine, equine, canine, lupine, feline, murine, ovine, avian, piscine, caprine, corvine, acrine, or delphine. The subject may be a human. The subject may be a human infected by bacteria.

Where the polynucleotide-templated metal nanocluster is to be used on a surface, the polynucleotide-templated metal nanocluster may be formulated as a spray, gel, wipes, soap or liquid formulation.

There is also provided an antimicrobial agent comprising a polynucleotide-templated metal nanocluster. The antimicrobial agent may be an antibacterial agent. The polynucleotide-templated metal nanocluster may be one as mentioned above. The polynucleotide-templated metal nanocluster may inhibit bacterial growth and/or bacterial toxin production. The polynucleotide-templated metal nanocluster in the antibacterial agent may emit a fluorescence colour.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1A is a graph showing the emission colour ratio as a function of reaction time based on C10-templated silver nanoclusters. The circular dichroism spectrum showing the structure adopted by the DNA template surrounding the silver nanoclusters is also shown below the graph. FIG. 1B shows the same as those in FIG. 1A but based on C20-templated silver nanoclusters. FIG. 1C shows the same as those in FIG. 1A but based on C10T10-templated silver nanoclusters. In all of the graphs, the corresponding fluorescence colours of the samples with the corresponding time intervals are shown as photographic images in the inset of each graph.

FIG. 2A is a bar chart comparing the potency of (from left to right) a control, silver nanoparticles, C10-templated silver nanoclusters, C20-templated silver nanoclusters, C10T10-templated silver nanoclusters and silver nitrate in inhibiting the growth of Pseudomonas aeruginosa. FIG. 2B is a photograph of P. aeruginosa on an agar plate with strips of filter paper containing C20-templated silver nanoclusters, C10-templated silver nanoclusters and control.

FIG. 3A is a photograph of the experimental setup showing the relative effectiveness of silver nanoparticles, C10-templated silver nanoclusters and C20-templated silver nanoclusters to prevent the production of C₁₂HSL toxin at different stated concentrations. Green spots (circled in FIG. 3A) below the P. aeruginosa bacteria culture indicate the presence of C₁₂HSL. FIG. 3B is a diagram showing the effectiveness of silver nanoparticles, C10-templated silver nanoclusters and C20-templated silver nanoclusters in inhibiting the production of the toxin pyocyanin by P. aeruginosa. Only the sample in the top right hand corner (900 nM of C20-templated silver nanoclusters) is colourless, the remaining samples are green-blue in colour.

FIG. 4A is a graph of cell viability as a function of the concentration of the Ag⁺ containing species. The image below the graph is a photograph of the WST-1 assay for quantifying the cytotoxicity of C-20 templated silver nanoclusters and silver nitrate against human A549 cells.

FIG. 4B is a graph showing the percentage of human A549 cells killed by the silver containing species against the percentage of P. aeruginosa growth inhibited. The bottom-left of the graph represent low cytotoxicity and low antibacterial potency, while the top-right represents the other extreme.

FIG. 5A is a plot of normalized bacteria count (by measuring the optical density of the medium at 600 nm) against concentration of cyan-fluorescent C20-templated silver nanoclusters to determine the MIC₅₀ value of silver nanoclusters against Burkholderia cenocepacis. FIG. 5B is the same plot as FIG. 5A but to determine the MIC₅₀ value of silver nanoclusters against P. aeruginosa. The photos in the inset of the individual plots show the appearance of bacterial cell cultures containing different concentrations of the silver containing species, reflective of the extent of bacterial growth in them.

### EXAMPLES

Non-limiting examples of the invention will be further described in greater detail by reference to specific Examples, which should not be construed as in any way limiting the scope of the invention.

All materials including DNA, AgNO₃ and NaBH₄ were purchased from Sigma Aldrich (of Missouri of the United States of America) and used directly without further purification. The DNA sequences were made-to-order. All solutions were made up with de-ionised water and all reactions was carried out in the same medium.

### Example 1

Controlled-Release Formulation of Ag⁺ from DNA-Templated Silver Nanoclusters Monitored by Fluorescent Colour Changes

By changing the DNA sequence to form different supramolecular structures to encapsulate silver nanoclusters (AgNCs) within the DNA templates, the controlled release rate of Ag⁺ from the ultrasmall AgNCs' surface can be tuned. In principle, a DNA template with well-defined structures can securely encapsulate the quantum-sized AgNCs from natural oxidation, giving a slow rate of spontaneous oxidative Ag⁺ release.

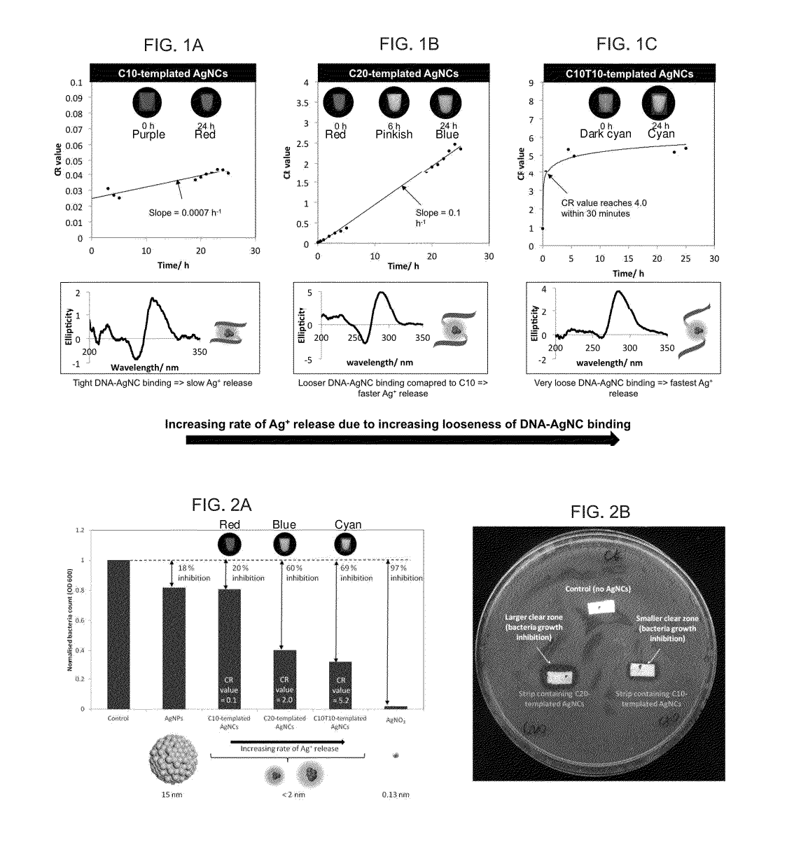

FIG. 1 illustrates how different DNA structures arising from varying the DNA sequence affected the Ag⁺ release rate. Three DNA sequences were tested in this study: C10 (a 10-base homocytosine DNA), C20 (a 20-base homocytosine DNA) and C10T10 (DNA having 10 consecutive cytosines followed by 10 consecutive thymines). In order to form the DNA-templated silver nanoclusters, a typical synthesis of is as follows: AgNO₃ (4.5 uL, 2.00 mM) was added to the respective DNA template as mentioned above (9.0 uL, 0.10 mM) followed by water (32 uL) and vigorously mixed by vortexing for 30 seconds. After standing for 20 minutes, a freshly-prepared aqueous solution of NaBH₄ (4.5 uL, 2.00 mM) was added and mixed by vortexing for a further 30 seconds. The fluorescence of the AgNC solution (50.0 uL) was monitored at regular time intervals, and kept at room temperature in the dark to prevent photo-reduction of Ag⁺ between fluorescence measurements.

The Circular Dichroism (CD) spectrum in FIG. 1A and FIG. 1B showed that both the C10 and C20 homocytosine DNAs were able to adopt the well-defined structures in solution known as i-motifs which can securely encapsulate AgNCs within. The longer DNA chain, i.e., C20 template had a greater degree of molecular flexibility than C10, thereby allowing the AgNCs to undergo a faster rate of spontaneous Ag⁺ release than the C10-templated-AgNCs. On the other hand, the presence of thymine bases in C10T10 caused the DNA to form less well-defined i-motif structures as compared to the C20 sequence of the same length (see CD spectrum in FIG. 1C), resulting in a looser DNA-AgNC binding and hence the fastest rate of Ag⁺ release was observed from C10T10-templated AgNCs amongst the three cytosine-containing DNA sequences tested in this study.

Due to the intrinsic photoluminescent properties of these color changing AgNCs, the extent of Ag⁺ controlled-release following the spontaneous oxidative Ag⁺ release process (from red to cyan) can be easily tracked. When the red-fluorescent AgNCs react with oxygen, Ag⁺ was released. While some of these Ag⁺ remained free-floating in solution, others can grow on the existing red-emitting AgNCs (emitting at a wavelength of 640 nm) to form larger sub-2 nm green- (emitting at 540 nm) and blue- (emitting at 490 nm) fluorescent species. This caused the fluorescence colour of the AgNC solution to turn increasingly cyan with time. The fluorescence colour change accompanying Ag⁺ release can be quantified by the emission colour ratio (CR), which is defined by the quotient between the sum of green and blue fluorescence intensities to the red fluorescence intensity at any instant, or

$$C R value = \frac{\begin{matrix}Green ( {λ}_{nm} = 540 nm ) + \\ Blue ( {λ}_{nm} = 490 nm ) fluorescence intensity\end{matrix}}{Red \{ {λ}_{nm} = 640 nm ) fluorescence intensity} .$$

Based on this definition, the release rate of Ag⁺ can be determined by the slope of the CR-versus-time plot. FIG. 1A shows that C10-templated AgNCs, which exhibit red fluorescence after 24 hours, gave the slowest Ag⁺ release rate of 0.0007 CR/h. FIG. 1B shows that C20-templated AgNCs has faster Ag⁺ release, occurring at a rate of 0.1 CR/h. FIG. 1C shows that C10T10-templated AgNCs are the fastest Ag⁺ release formulation, where the CR value reached 4.0 within 30 minutes to achieve a cyan-fluorescent solution. Therefore, the rate of spontaneous oxidative Ag⁺ release from the DNA-templated AgNCs followed the order of C10T10>C20>C10.

Hence, by changing the structure of DNA sequences, tuning and fluorescence monitoring (via the red to cyan colour change) of the Ag⁺ release rate from DNA-templated AgNCs can be easily achieved.

### Example 2

### Tunable Antimicrobial Activity of DNA-Templated AgNCs Against Multidrug-Resistant Bacteria

Here, the release rate of Ag⁺ from the different DNA-templated AgNCs is related to its antimicrobial potency to inhibit the bacterial growth. This Example demonstrates that DNA-templated AgNC solutions which gave a larger CR value are more effective in inhibiting bacterial growth than those with smaller CR values (at a fixed sampling time) due to the presence of more Ag⁺—the bioactive antibacterial species.

To demonstrate this correlation, AgNCs templated by C10, C20 and C10T10 were first synthesized and then mixed with the growing cell cultures of the multidrug-resistant bacteria P. aeruginosa in solution overnight at 37° C. under contact shaking of 250 rpm. C10T10-templated AgNCs (CR value=5.2) was found to be the most effective antimicrobial AgNC formulation, which is able inhibit up to 69% of the P. aeruginosa from growing (FIG. 2A, 5^{th} sample from the left), followed by the C20- (CR value=2.0) and C10- (CR=0.1) with 60% and 20% bacteria inhibition activities respectively (FIG. 2A, 3^{rd} and 4^{th} samples from the left). These results suggest that DNA-AgNC solutions bearing CR values greater than 2.0 (cyan-fluorescent) are able to release Ag⁺ at sufficiently fast rates to be effective antibacterial agents.

Other than DNA-based surface chemistry, the size-dependent controlled-release of Ag⁺ from the DNA-templated AgNCs (sub-2nm) were also compared with the citrate-capped silver nanoparticles (AgNPs, 15 nm) and AgNO₃ (free flow of Ag⁺) against bacterial growth under the same experimental conditions. AgNPs were synthesized based on the Turkevich method whereby an aqueous solution of trisodium citrate was brought to boil, followed by addition of AgNO₃ aq solution (310 μM). The reaction was then boiled till a yellow solution was obtained. For the AgNO₃, 180 μM of AgNO₃ was used for this example.

FIG. 2A shows that all of the three tested ultrasmall AgNCs are more effective (due to larger surface area for releasing Ag⁺) than the larger AgNPs which only inhibited 18% of bacterial growth (2^{nd} sample from the left in FIG. 2A). While the AgNO₃ that releases all the toxic Ag⁺ to the sample could inhibit up to 97% bacterial growth (rightmost sample in FIG. 2A), its high dosage direct application is extremely toxic to the human cells as well (see cytotoxicity test results of AgNO₃ in Example 4 further below).

To show the potency of AgNCs for wound dressing application, the Kirby-Bauer bacterial zone inhibition assay was used to test the antimicrobial activity of AgNCs doped onto filter papers. The AgNC-containing strips of filter paper were prepared by pre-soaking in the respective AgNCs solution for an hour, followed by allowing them to dry in air. The process was repeated 4 times to ensure good impregnation of AgNCs within the filter paper. The multidrug-resistant bacteria P. aeruginosa was allowed to grow overnight at 37° C. before the photograph shown in FIG. 2B was taken. FIG. 2B shows that a larger clear zone (no bacteria growth) was observed around the filter papers with cyan-fluorescent C20-templated AgNCs than that with the red-fluorescent C10-templated AgNCs. This result confirms the biological relevance of the disclosed Ag⁺ control-release formulation, where AgNCs templated by a DNA sequence (e.g., C20) that allow a faster rate of Ag⁺ release brings about greater antibacterial potency.

Additionally, the colour change properties of AgNCs from red to cyan due to the oxidative release of Ag⁺ can allow one to potentially track the extent of bacterial cell death due to the interactions of bacteria and Ag⁺. Oxidation of red-emitting AgNCs release Ag⁺ to form green- and blue-emitting AgNCs. In the presence of bacteria cells, these Ag⁺-bacteria interactions would result in removal of Ag⁺ from the AgNCs, causing the fluorescence to turn back to red. Therefore, a red-shift in AgNC fluorescence (or decrease in CR values) is expected to be observed as more bacteria interact with Ag⁺. Hence, DNA-templated AgNCs also provide a unique and unprecedentedly convenient means to assess the extent of bacterial cell death for a diverse range of antibacterial applications.

In summary, Examples I and II show the easy tuning of Ag⁺ controlled release rate and their antibacterial potency by simply changing the sequence of the DNA template of the AgNCs. The resulting fluorescence colour of AgNCs provides a convenient and unambiguous means of assessing their antibacterial potency whose effects are general towards bacteria grown in solution and on surfaces, allowing easy adaptation of AgNCs for diverse applications.

### Example 3

### AgNCs are Effective Inhibitors of Bacteria Toxin Production

Inhibition of bacterial growth alone is insufficient to offer complete protection against the dangers associated with bacterial proliferation. During the growth process, bacteria produce a host of signaling and communication molecules to coordinate their growth in a process known as quorum sensing. Most of these chemicals are toxic, enabling the invading bacteria species to out-compete existing bacterial colonies already present, or attack the host organism to ensure more effective colonization. Herein, the first silver-based antibacterial agent that can interrupt quorum sensing and inhibit the toxin production, e.g., pyocyanin from the superbug P. aeruginosa, is shown.

The ability of AgNCs to inhibit an essential quorum sensing signal C₁₂HSL produced by P. aeruginosa for controlling its population density and production of dangerous toxins known as virulence factors is investigated here.

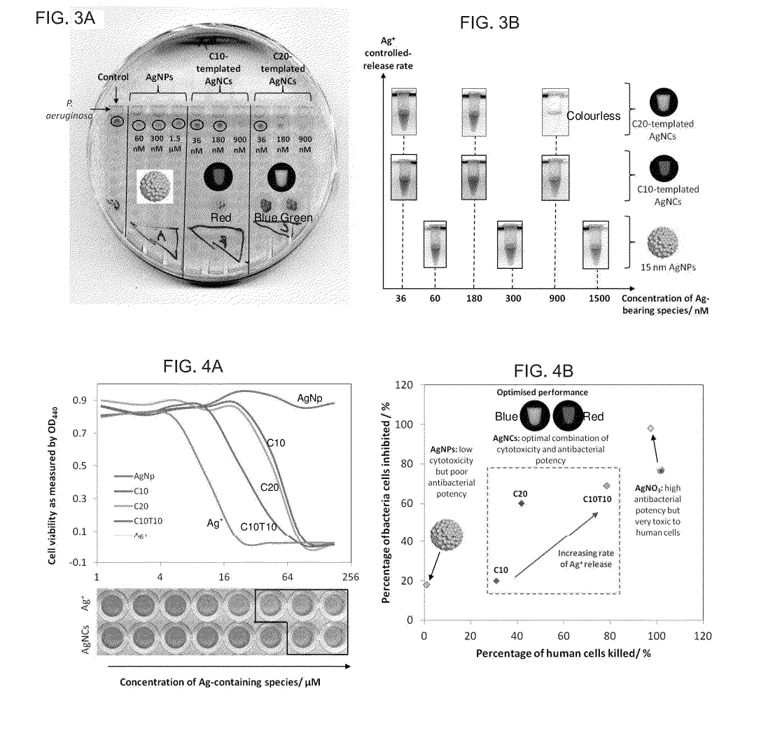

C₁₂HSL itself is also cytotoxic, capable of disrupting mammalian cell processes such as DNA transcription (PLoS Pathogens, 2012, 8, e1002853). Hence, the ability of the DNA-templated AgNCs to inhibit its production prevents the superbug from reaching population densities dangerous to humans, while limiting bacterial virulence at the same time. To experimentally show this unique ability of the DNA-templated AgNCs, P. aeruginosa was mixed with AgNCs and grown at the top of agar strips containing another bacteria strain (Agrobacterium tumefaciens) grown at regular intervals which act as an indicator for C₁₂HSL (FIG. 3A). As P. aeruginosa produced C₁₂HSL, the chemical diffuses down the strip and caused the A. tumefaciens bacteria to turn green. Hence, a more intense green spot signified that more C₁₂HSL was produced. From FIG. 3A, it can be observed that the fast Ag⁺ releasing formulation of C20-templated AgNCs was able to inhibit more C₁₂HSL production than the slower Ag⁺-releasing formulation of C10-templated AgNCs, as a more obvious green spot can be seen for the latter at 180 nM than for the former at the same concentration. At 900 nM, AgNCs templated by both C10 and C20 showed complete inhibition of C₁₂HSL production, with no green colouration observed at all. In contrast, AgNCs, which showed a very slow rate of Ag⁺ production, do not cause significant C₁₂HSL inhibition as compared to the control.

The C₁₂HSL quorum sensing molecule also regulates and controls the production of one of P. aeruginosa's most powerful and dangerous toxins, called pyocyanin. This molecule is particularly dangerous to humans as it is highly lethal to mammalian cells, and is responsible for large numbers of deaths arising from P. aeruginosa in hospitals worldwide. Therefore, the ability to prevent pyocyanin production can potentially save countless lives. To illustrate the pyocyanin inhibition properties of the DNA-templated AgNCs, the P. aeruginosa together with C10- or C20-templated AgNCs was grown in cell cultures. Being a blue pigment, the production and presence of pyocyanin can be detected by a blue colouration of the bacterial culture. From FIG. 3B, C20-templated AgNCs at a concentration of 900 nM were able to completely inhibit the production of the pyocyanin toxin, giving a clear solution. However, C10-templated AgNCs at the same concentration did not show significant inhibition of its production. For the larger 15 nm AgNPs, no toxin inhibition was observed up to 1.5 μM. Hence, this example shows the unique properties of DNA-templated AgNCs in inhibiting bacteria toxin production as compared to other AgNPs-based products.

The ability to control the rate of Ag⁺ controlled-release from AgNCs by using different DNA sequences also equates to their varying efficacies in preventing bacterial toxin production. This novel property of DNA-templated AgNCs is significant as it represents the first-ever reported case of preventing bacterial toxin production by a silver-based formulation, thereby ensuring an all-round protection against bacteria by ensuring that no toxins are produced by any bacteria still present.

### Example 4

Optimal Controlled-Release AgNCs Formulation with High Antibacterial Potency and Low Cytotoxicity

Here, the eukaryotic cytotoxicity of the DNA-templated AgNC on human cells will be investigated. A global comparison of the antibacterial potencies and cytotoxicity of AgNCs with AgNO₃ and AgNPs will also be demonstrated.

The cytotoxicity of the silver-containing species were tested against human A549 cells (from the lungs) using the WST-1 Assay (manufactured by Roche). The silver-bearing species were diluted to different extents and allowed to incubate with the human cells at 37° C. for 4 hours. In the presence of dead cells, the enzymatic assay gave a pink coloured solution (as shown by the box in FIG. 4A), while the living cells gave a brown solution (cells that are outside the box region in FIG. 4A).

The percentage of human dead cells can be quantified spectroscopically by measuring the optical density of the solution at a wavelength of 440 nm. To examine the antimicrobial potency and cytotoxicity of silver in different formulations (AgNPs, DNA-templated AgNCs and AgNO₃), the percentage of bacteria cells inhibited (y-axis representing the antibacterial potency) was plotted against the percentage of human A549 cells killed (x-axis representing the eukaryotic cytotoxicity) in FIG. 4B. Here, the relative percentages of human A549 cells killed were compared at a concentration of 44 μM for all silver-containing species.

It is noticed that the AgNPs and AgNO₃ take the extreme positions on the plot, which either possessed low antibacterial potency (AgNPs) or high cytotoxicity (AgNO₃). For example, AgNPs with slow Ag⁺ release was able to inhibit only 20% of the harmful bacteria although it has low cytotoxicity. On the other extreme, the powerful antibacterial property of AgNO₃ with no controlled-release of Ag⁺ was extremely harmful to the human cells with high toxicity. It is clear in FIG. 4B that the disclosed DNA-templated AgNCs take the middle ground between AgNPs and AgNO₃. By tuning the DNA sequences, the optimized formulation of DNA-templated AgNC with high antimicrobial potential and low cytotoxicity can be achieved. For example, C20-templated AgNCs can inhibit up to 60% of the bacterial cell growth with cytotoxicity that is much lower than that for AgNO₃. These results show the uniqueness of current DNA-templated AgNCs as a novel class of Ag⁺ controlled-release materials with the ability to optimize the performance of antimicrobial activities combining the beneficial aspects of AgNPs with their low eukaryotic cytotoxicity and efficient antibacterial properties of Ag⁺.

### Example 5

### General Antibacterial Potency of DNA-Templated AgNCs Towards Superbugs

Superbugs are bacteria species that have developed drug resistance to a wide range of antibiotics, and are responsible for a large number of fatal human infections in hospitals. Treatment of superbug infection is painful, costly and difficult, often requiring combinations of multiple antibiotics and/or drug administration via injections. Hence, the generality of the antibacterial effects of DNA-templated AgNCs, as a controlled-release platform for the bioactive Ag+, makes them particularly valuable as an alternative material for preventing the growth and proliferation of these multidrug-resistant bacteria.

In previous examples, C20-templated AgNCs has been identified as the optimum formulation for inhibiting bacteria P. aeruginosa with low cytotoxicity. In this example, C20-templated AgNCs was tested on another common multidrug-resistant bacteria, Burkholdera cenocepacia, to demonstrate that their antimicrobial potency is as effective as the best existing antibiotics.

The effectiveness of C20-templated AgNCs in inhibiting bacterial growth was determined by measuring the MIC₅₀ value of AgNCs at which concentration prevents 50% of the bacteria from growing (relative to a control containing no AgNCs). A series of bacteria cultures was prepared containing increasing concentrations of C20-templated AgNCs. The bacteria concentration in each culture after overnight incubation at 28° C. at constant agitation of 250 rpm was determined by measuring the optical density of the culture solution at a wavelength of 600 nm. A more concentrated bacteria solution gives a higher optical density reading, translating to a more turbid (less clear) solution when seen with the naked eye.

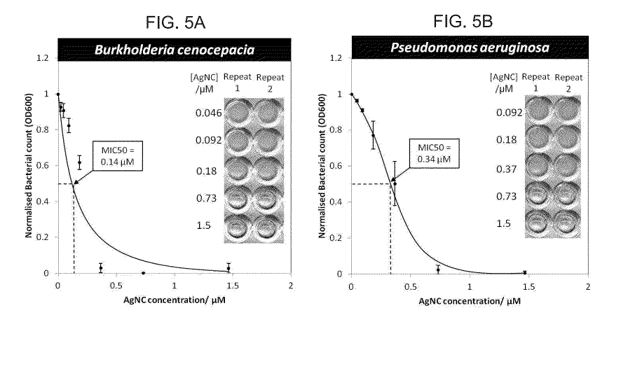

In FIG. 5A, the growth media containing a higher concentration of C20-templated AgNCs were found to contain lesser bacteria, giving rise to clearer solutions (inset photos) and a smaller optical density reading. Using these plots, the MIC₅₀ value of C20-templated AgNCs was found to be approximately 0.14 μM for B. cenocepacia in FIG. 5A and 0.34 μM for P. aeruginosa in FIG. 5B, respectively. The concentrations of C20-templated AgNCs were calculated based on the concentration of the precursor AgNO₃ used (180 μM), with an average of 8 Ag atoms per nanocluster as determined from ESI mass spectroscopy (results not shown). Comparatively, the MIC₅₀ value of ciprofloxacin, one of the most potent antibiotics against these superbugs, is 0.36 μM against B. cenocepacia (J. Antimicrob. Chemother., 2002, 50, 265-269) and 0.27 μM against P. aeruginosa (J. Antimicrob. Chemother., 1999, 43, 345-349). These findings confirmed that powerful antibacterial effects of the DNA-templated AgNCs are comparable with the most effective antibiotics available in the market. The cytotoxicity studies in Example 4 also revealed that the MIC₅₀ value of C20-templated AgNCs against human A549 cells was 50 μM, indicating that their antibacterial effects are more than 100 times stronger than their cytotoxicity to human cells, which confirms the optimized formulation of DNA-templated AgNCs as a safe and effective antibacterial agent.

In conclusion, DNA-templated AgNCs has been shown to be easily customizable. The Ag⁺ release rate from these sub-2 nanometer fluorescent species can be tuned by simply changing the DNA sequence. Furthermore, the rate of Ag⁺ release can be easily and unambiguously observed by a change in the AgNC fluorescence colour (Example 1). A cyan-fluorescent DNA-templated AgNC solution, bearing CR values of at least 2.0, showed fast Ag⁺ release and strong bacterial growth inhibition properties (Example 2), while preventing bacterial toxin production (Example 3). Despite these strong antibacterial properties, these DNA-templated AgNCs possess low eukaryotic cytotoxicity, making them an optimal new-generation of antibacterial controlled-release agents (Example 4). Finally, the antibacterial effects of DNA-templated AgNCs are general towards different species of multidrug-resistant bacteria and are as powerful as the best available antibiotics known today, enabling them to function as an alternative antibacterial agent (Example 5).

### APPLICATIONS

The disclosed polynucleotide-templated metal nanoclusters can be used in a number of applications that result from the microbial properties of the metal nanoclusters. The disclosed polynucleotide-templated metal nanoclusters can be used as an alternative option to conventional antibiotics. The disclosed polynucleotide-templated metal nanoclusters are able to bridge the gaps between metal nanoparticles and metal salt solutions and bring about the desirable traits of potent antibacterial properties with low cytotoxicity.

The disclosed polynucleotide-templated metal nanoclusters are simple and fast to prepare. The low cost of production can enable affordable large-scale implementation.

The release rate of the metal ion from the disclosed polynucleotide-templated metal nanoclusters can be tuned by simply changing the structure of the polynucleotide.

The photoluminescent properties that are responsive to the release rate of the metal ion from the polynucleotide-templated metal nanoclusters can be used as a fluorescent indicator of antimicrobial potency.

The disclosed polynucleotide-templated metal nanoclusters are effective inhibitor of microbial toxin production.

The disclosed polynucleotide-templated metal nanoclusters are effective microbial agents (such as antibacterial agents) for diverse applications in consumer-care and medical industries, such as in wound dressing, antiseptic spray, antimicrobial surfaces, etc.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for inhibiting the growth of or killing microorganism that is present on a surface comprising the step of administering a polynucleotide-templated metal nanocluster to said surface.

2. The method of claim 1, wherein said microorganism is a bacteria selected from the group consisting of a Gram positive bacteria and a Gram negative bacteria.

3. The method of claim 2, wherein said Gram negative bacteria is Pseudomonas aeruginosa or Burkholdera cenocepacia.

4. The method of claim 1, wherein said polynucleotide is a DNA, RNA or mixtures thereof.

5. The method of claim 4, wherein said DNA is single-stranded DNA or double-stranded DNA.

6. The method of claim 5, wherein said DNA is selected from the group consisting of homocytosine single-stranded DNA sequence, homoadenine single-stranded DNA sequence, homothymine single-stranded DNA sequence, homoguanine single-stranded DNA sequence, 5′-AGGTCGCCGCCC-3′(SEQ ID NO:1), 5′-AATTCCCCCCCCCCCCAATT-3′ (SEQ ID NO:2), i-motif (dTA₂C₄)₄ (SEQ ID NO:3), i-motif (dC₄A₂)₃C₄ (SEQ ID NO:4), 5′-CCCTTTAACCCC-3′ (SEQ ID NO:5), 5′-CCCTCTTAACCC-3′(SEQ ID NO:6), 5′-CCCTTAATCCCC-3′ (SEQ ID NO:7), 5′-CCTCCTTCCTCC-3′(SEQ ID NO:8), 5′-CCCTAACTCCCC-3′(SEQ ID NO:9), 5′-CCCACCCACCCTCCCA-3′(SEQ ID NO:10), dT₄C₄T₄ (SEQ ID NO:11), dsDNA with a C₆ loop and mixtures thereof.

7. The method of claim 4, wherein the length of said polynucleotide is at least 2-nucleobases.

8. The method of claim 1, wherein the metal of the metal nanocluster is selected from the group consisting of silver, gold, copper, palladium, ruthenium, rhodium, osmium, iridium, platinum, rhenium, mixtures and complexes thereof.

9. The method of claim 8, wherein the metal of the metal nanocluster is silver.

10. The method of claim 1, wherein the nanocluster has a particle size of 0.01 nm to 2 nm, 0.01 nm to 1.5 nm, 0.01 nm to 1 nm, 0.01 nm to 0.5 nm, 0.01 nm to 0.1 nm, 0.1 nm to 2 nm, 0.5 nm to 2 nm, 1 nm to 2 nm or 1.5 nm to 2 nm.

11. The method of claim 1, comprising administering said polynucleotide-templated metal nanocluster at a concentration of at least 150 nM.

12. The method of claim 1, wherein said polynucleotide-templated metal nanocluster inhibits microbial toxin production.

13. The method of claim 1, wherein said surface is a skin surface of a human or animal body.

14. The method of claim 1, wherein said surface is a surface of an object.

15. An antimicrobial agent comprising a polynucleotide-templated metal nanocluster.

16. The antimicrobial agent of claim 15, wherein said polynucleotide-templated metal nanocluster inhibits microbial growth.

17. The antimicrobial agent of claim 15, wherein said polynucleotide-templated metal nanocluster inhibits microbial toxin production.

18. The antimicrobial agent of claim 15, wherein said polynucleotide-templated metal nanocluster emits a fluorescence colour.
